(19) United States
(12) Patent Application Publication
(10) Pub. No.: US 2007/0291251 A1
(43) Pub. Date: Dec. 20, 2007

(54) Optical Analysis System With Background Signal Compensation

(75) Inventors: Wouter Harry Jacinth Rensen, Eindhoven (NL); Bernardus Leonardus Gerardus Bakker, Eindhoven (NL); Michael Cornelis Van Beek, Eindhoven (NL)

Correspondence Address: PHILIPS INTELLECTUAL PROPERTY & STANDARDS, 595 MINER ROAD, CLEVELAND, OH, 44143, US

(73) Assignee: Koninklijke Philips Electronics N.V., Eindhoven (NL)

(21) Appl. No.: 11/574,181

(22) PCT Filed: Aug. 24, 2005
(86) PCT No.: PCT/IB05/52765

§ 371 Date: Feb. 23, 2007

(30) Foreign Application Priority Data
Aug. 27, 2004 (EP) 04104123.7

(51) Int. Cl. G01N 33/48; G01N 21/00; G06F 19/00

(52) U.S. Cl. 356/39; 356/73; 702/85

(57) ABSTRACT

The invention provides an optical analysis system for efficient compensation of spectroscopic broadband back-ground, such as spectroscopic fluorescence background or background signals that are due to the dark current of a detector. The optical analysis system effectively provides multivariate optical analysis of a spectroscopic signal. It provides wavelength selective detection of various spectral components that are indicative of a superposition of spectroscopic peaks or bands and their broadband background. Additionally, the optical analysis system is adapted to acquire spectral components that predominantly correspond to the broadband background of the spectroscopic peaks or bands. Wavelength selective selection of various spectral components is performed on the basis of reconfigurable multivariate optical elements or on the basis of a position displacement of a spatial optical transmission mask.

The present invention relates to the field of optical spectroscopy.

Spectroscopic techniques are widely used for determination of the composition of a substance. By spectrally analyzing an optical signal, i.e. a spectroscopic optical signal, the concentration of a particular compound of the substance can be precisely determined. The concentration of a particular substance is typically given by an amplitude of a principal component of an optical signal.

U.S. Pat. No. 6198,531 B1 discloses an embodiment of an optical analysis system for determining an amplitude of a principal component of an optical signal. The known optical analysis system is part of a spectroscopic analysis system suited for, e.g., analyzing which compounds are comprised at which concentrations in a sample. It is well known that light interacting with the sample carries away information about the compounds and their concentrations. The underlying physical processes are exploited in optical spectroscopic techniques in which light of a light source such as, e.g., a laser, a lamp or light emitting diode is directed to the sample for generating an optical signal which carries this information.

For example, light may be absorbed by the sample. Alternatively or in addition, light of a known wavelength may interact with the sample and thereby generate light at a different wavelength due to, e.g. a Raman process. The transmitted and/or generated light then constitutes the optical signal which may also be referred to as the spectrum. The relative intensity of the optical signal as function of the wavelength is then indicative for the compounds comprised in the sample and their concentrations.

To identify the compounds comprised in the sample and to determine their concentrations the optical signal has to be analyzed. In the known optical analysis system the optical signal is analyzed by dedicated hardware comprising an optical filter. This optical filter has a transmission which depends on the wavelength, i.e. it is designed to weight the optical signal by a spectral weighting function which is given by the wavelength dependent transmission. The spectral weighting function is chosen such that the total intensity of the weighted optical signal, i.e. of the light transmitted by the filter, is directly proportional to the concentration of a particular compound. Such an optical filter is also denoted as multivariate optical element (MOE). This intensity can then be conveniently detected by a detector such as, e.g., a photodiode. For every compound a dedicated optical filter with a characteristic spectral weighting function is used. The optical filter may be, e.g., an interference filter having a transmission constituting the desired weighting function.

Typically, the principal component comprises a positive part and a negative part. Therefore, a part of the optical signal is directed to a first filter which weights the optical signal by a first spectral weighting function corresponding to the positive part of the principal component, and a further part of the optical signal is directed to a second filter which weights the optical signal by a second spectral weighting function corresponding to the negative part of the principal component. The light transmitted by the first and second filters is then separately detected by a first and a second detector, respectively. The two signals obtained by the two detectors are then subtracted, resulting in a signal with an amplitude corresponding to the concentration of a dedicated compound of the sample.

In this way, instead of the entire spectrum, only a single signal that is proportional to a specific compound of the sample is detected. Hence, a rather expensive charge coupled device (CCD)—camera can be effectively replaced by low-cost light sensitive detectors, such as e.g. semi-conductor based photodiodes.

In many spectroscopic analysis systems elastically scattered radiation as well as dark current of the detector may give rise to appreciable background signals that are superimposed to the intrinsic spectroscopic signal. Typically, spectroscopic signals that have to be analyzed feature relatively narrow peaks in the spectrum compared to the broadband fluorescence or dark current background. Generally, a reliable and sufficient spectroscopic analysis requires effective elimination of broadband background signals.

This can for example be provided by filtering of slowly varying signal components of a spectrum. However, by making use of MOE's only a single signal rather than the entire spectrum is detected. Consequently, a filtering of slowly varying spectral components cannot be performed in a straightforward way. However, background compensation is a necessary step of spectroscopic signal analysis and it also has to be applied on spectroscopic analysis based on multivariate optical analysis.

The advantages of a background compensation scheme are obvious, when for example the background is subject to modifications which might easily occur in the framework of spectroscopic analysis of biological tissue. In particular, when spectroscopic analysis is applied to a variety of different biological tissues featuring different optical properties, a fluorescence background may strongly depend on the type of the biological tissue. Moreover, other effects like scattering of light in a light guiding arrangement providing transmission of collected optical signals to a spectroscopic analysis system may also have a major impact on the background level. Also, when the background is non uniform, i.e. the fluorescence or dark current is non uniform over a large spectral range, subtracting a constant fluorescence and dark current background would severely falsify the spectroscopic signal to a large extent.

The present invention therefore aims to provide background correction of an optical spectrum on the basis of multivariate optical elements.

The present invention provides an optical analysis system for determining a principal component of an optical signal. The optical analysis system comprises a dispersive optical element, spatial light manipulation means, a detector and processing means. The dispersive optical element serves to spatially separate spectral components of the optical signal in a first direction. Typically, the dispersive optical element is implemented as a grating or prism. The spatial light manipulation means serve to at least partially transmitting at least a first spectral component of the optical signal at a first time and for transmitting at least a second spectral component of the optical signal at a second time.

The at least first and second transmitted spectral components of the optical signal are then detected by means of the detector. Hence, the at least first and second transmitted spectral components are sequentially detected at the first and the second time. In this way the detector serves to detect at least a first and at least a second transmitted spectral component of the optical signal in a sequential way. The processing means are adapted to perform a correction on the optical signal on the basis of the at least first and second detected spectral components. In a strict sense the processing means are adapted to perform the correction on electrical signals that are obtained from the detector in response to detect the at least first and second transmitted spectral components.

The invention is particularly based on the assumption, that the optical signal comprises a broadband background and narrow band spectroscopic peaks that are relevant for the determination of the principal component of the optical signal. Furthermore, since the wavelength of compound specific spectroscopic peaks is known, the spatial light manipulation means may serve to selectively transmit only a distinct spectral band that substantially comprises a particular spectroscopic peak. For example, the first transmitted spectral component of the optical signal may correspond to a narrow Raman band.

Actually the corresponding signal being detected by means of the detector at the first time comprises a contribution from the broadband background as well as a contribution from the narrow Raman band. Hence, the first spectral component of the optical signal that is transmitted by the spatial light manipulation means at a first time represents a superposition of a broadband background and a narrow band spectroscopic signal.

In order to decompose and to separately resolve the Raman and the background contribution of the transmitted first spectral component of the optical signal, the background level has to be determined and subtracted from the detected first spectral component. Therefore, the spatial light manipulation means provide transmitting of at least a second spectral component of the optical signal at a second time. Here, the spatial light manipulation means are configured in such a way, that the second spectral component of the optical signal does not comprise the desired Raman contribution but exclusively corresponds to a background signal that is comparable to the background contribution of the first spectral component being transmitted by the spatial light manipulation means at the first time. Typically, the second spectral component is only slightly shifted compared to the first spectral component in such a way, that it covers a spectral band that is adjacently located to the first spectral band of the first spectral component.

According to a further preferred embodiment of the invention, the spatial light manipulation means are shiftable along the first direction and further comprise a fixed transmission aperture. In this basic embodiment the spatial light manipulation means can be effectively realized in form of a slit aperture that is shiftable along the first direction, i.e. along the direction of spatial decomposition of the optical signal or the direction of the spectrum evolving from the dispersive optical element. For example, the spatial light manipulation means can be realized as a spatial transmission mask featuring a plurality of slit apertures, where each slit corresponds to a distinct Raman band. In this way the spatial transmission mask serves to transmit a plurality of Raman peaks whereas other spectral components of the optical signal are blocked. Since the transmitted Raman peaks typically comprise a significant contribution of fluorescence and noise background, the spectroscopic contribution, e.g. the Raman contribution of the transmitted spectral components has to be extracted. By slightly shifting the spatial light transmission mask in the direction of the spectrum of the optical signal, such that the spectroscopic peaks are substantially blocked by the spatial light transmission mask and that a neighboring background level is transmitted, the evolving signal that can be consequently detected by means of the detector may merely corresponds to the background contribution of the previously recorded spectroscopic signal. By mutually subtracting these two sequentially recorded signals the spectroscopic contribution can be sufficiently resolved.

Alternatively, instead of shifting the spatial light manipulation means also the dispersive optical element might be subject to e.g. rotation, such that the transverse position of the spectroscopic peaks can be shifted with respect to the slit apertures of the spatial light manipulation means. In this way either the spatial light manipulation means or the spectrum in its entirety has to be, e.g. transversally, shifted. This mutual shifting can in principle be realized by a manifold of optical arrangements, that provide changing the direction of the incident optical signal by appropriately shifting or tilting of any of the involved optical components. It should only be guaranteed that during detection of the at least first transmitted spectral component the spatial position of spectroscopic peaks of the optical signal substantially match the position of various apertures of the spatial light manipulation means.

In contrast, during acquisition of the at least second transmitted spectral component of the optical signal, the spectroscopic peaks of the optical signal should be blocked by means of the spatial light manipulation means and only adjacently located spectral bands corresponding to background fluorescence or background noise should be transmitted by means of the spatial light manipulation means.

According to a further preferred embodiment of the invention, the spatial light manipulation means comprise a reconfigurable spatial light modulator. By implementing the spatial light manipulation means as a reconfigurable spatial light modulator, the spatial light manipulation means can be rigidly fixed in the optical analysis system. Consequently, the spatial light manipulation means do no longer have to be shifted with respect to the spectrum of the optical signal but transmission of a first spectral component and a subsequent transmission of a second neighboring spectral component of the optical signal can be effectively realized by reconfiguring of the spatial light modulator.

For example, the spatial light modulator can be effectively realized by an array of individually switchable liquid crystal cells that are positioned between crossed polarizers. The single liquid crystal cells can be electrically switched in order to modify the polarization direction of the incident light. In combination with the crossed polarizer arrangement a switchable, hence reconfigurable, spatial transmission mask can be effectively realized. In this way by appropriately controlling the operation of the reconfigurable spatial light modulator, specific spectral bands can be selectively transmitted or blocked. Hence, the reconfigurable spatial light modulator serves to transmit the at least first spectral component of the optical signal at the first time and subsequently serves to transmit the at least second spectral components of the optical signal at the second time. Realizing the reconfigurable spatial light modulator as e.g. a liquid crystal cell arrangement, selective transmission of first and second spectral components of the optical signal does not involve any mechanical movement or shifting.

According to a further preferred embodiment of the invention, the spatial light manipulation means further comprise an aperture that has at least a first slit. The width of this at least first slit is modifiable. By making the width of the transmissive aperture of the spatial light manipulation means configurable, the spatial light manipulation means can be individually adapted to a plurality of different spectral bands featuring spectroscopic peaks of different width. For example, the width of the transmission aperture can be modified in such a way that it only allows for transmission of a single spectroscopic peak. Hence, the at least first transmitted spectral component may therefore feature a very narrow spectral band.

In contrast, the width of the spatial light manipulation means' aperture may also provide transmission of a fairly broad spectral band, which is advantageous for acquisition of the background signal. Typically, the absolute intensity of the background signal is much larger than the absolute intensity of a distinct spectroscopic peak. Hence, by increasing the spectral band of a transmitted spectral component the background contribution of a detected signal significantly increases. Therefore, as an alternative to a shifting of the spatial light manipulation means, its aperture can be extended leading to a significant increase of the background contribution of the detected signal at the expense of the spectroscopic contribution. In this way the fairly broadband transmitted spectral component becomes predominantly representative of the background signal.

According to a further preferred embodiment of the invention, the spatial light manipulation means further comprise at least a second slit. This second slit also forms an aperture of the incident spectrally decomposed optical signal. The at least first and second slit apertures of the spatial light manipulation means are simultaneously shiftable along the first direction. Typically, the position of the at least first and second slit apertures of the spatial light manipulation means correspond to the transverse position of significant spectral bands of the optical signal. The at least first and second slit apertures provide transmission of corresponding spectroscopic peaks at the first time. Hence the signal acquired by the detector at the first time is representative of at least two spectroscopic peaks and associate background contributions. By simultaneously shifting the at least first and second slit apertures preferably by the same distance along the first direction, a corresponding second signal can be subsequently detected by means of the detector at the second time. This second signal may then be exclusively representative of the superimposed background noise of the at least first and second spectral components that were acquired at the first time.

According to a further preferred embodiment of the invention, the reconfigurable spatial light modulator is adapted to form a slit aperture that moves along the first direction. In this embodiment the reconfigurable spatial light modulator is driven in e.g. a scanning mode, i.e. the reconfigurable spatial light modulator serves to subsequently transmit a plurality of adjacent spectral bands of the entire spectrum. In this way a complete spectrum featuring spectral peaks and a broad fluorescence and/or dark current background is segmented into a plurality of contiguous spectral bands that are subsequently transmitted and recorded by means of the spatial light modulator and the detector.

The width of the various segments of the spectrum might be arbitrarily chosen and might be non uniform. Also the width of the aperture may dynamically change during a scan through the spectrum. By segmenting the entire spectrum into a small amount of broadband segments, such a scan can be performed in a relatively short time because of the rather small number of segments. This provides a rather rough estimation of the background level. Alternatively, such a spectral scan can also be based on a large number of narrow band spectral segments providing a more reliable determination of the background level at the expense of a larger time interval required for a scan of the entire spectrum.

Furthermore, the position of the at least first slit of the spatial light manipulation means may arbitrarily vary, e.g. on a periodical basis. In this way, different spectral bands of the spectrum may serve as a basis for background compensation which allows to account for a background that varies in time. Variation of the position of the at least first slit of the spatial light manipulation means can either be realized by shifting and/or reconfiguring the spatial light manipulation means.

According to a further preferred embodiment of the invention, the dispersive optical element and the spatial light manipulation means form a multivariate optical element. Preferably, the spatial light manipulation means are implemented as a liquid crystal light modulator or as a digital micro-mirror device (DMD). Hence, spectrally dispersing the incident optical signal and directing the evolving spectrum on spatial light manipulation means allows to selectively attenuate and to block dedicated components of the spectrum. The spatial light manipulation means may comprise different spatial light transmission sections, each of which providing selective transmission of distinct spectral components of the optical signal.

For example, the spatial light manipulation means feature a first and a second spatial light transmission mask effectively providing wavelength selective transmission that corresponds to positive and negative parts of a regression function, respectively. The multivariate optical element can either be realized by means of a fixed transmission mask that is particularly designed for analysis of a distinct compound. Alternatively, the spatial light manipulation means of the MOE can also be implemented as a reconfigurable arrangement, such as a liquid crystal light modulator or as a digital micro-mirror device, each of which providing reconfigurable and selective transmission of various spectral components of the optical signal.

According to a further preferred embodiment of the invention, the optical analysis system is adapted to provide non-invasive analysis of blood of a person. In this embodiment, the principal component to be determined by the optical analysis system is indicative of the concentration of a substance of the blood of the person. This substance may for example refer to one or more of the following analytes: glucose, lactate, cholesterol, oxy-hemoglobin and/or desoxy-hemoglobin, glycohemoglobin (HbAlc), hematocrit, cholesterol (total, HDL, LDL), triglycerides, urea, albumin, creatinin, oxygenation, pH, bicarbonate and many others.

In another aspect, the invention provides a method of performing a correction on an optical signal of an optical analysis system. The inventive method comprises the steps of spatially separating spectral components of the incident optical signal in a first direction by means of a dispersive optical element. In a further step the method comprises at least partially transmitting at least a first spectral component of the optical signal at a first time and subsequently at least partially transmitting at least a second spectral component of the optical signal at a second time. The partial transmitting of first and second spectral components is provided by spatial light manipulation means that are either non-reconfigurable and shiftable or reconfigurable and fixed in the optical analysis system.

The at least first and second transmitted spectral components of the optical signal are detected by means of a detector. Detection of the at least first and second spectral components is performed sequentially in order to clearly separate detection of the at least first and the at least second spectral components. After detection of the at least first and second transmitted spectral components, the detected spectral components are processed, preferably electronically, for performing the correction on the optical signal. Preferably, the at least first spectral component refers to a superposition of a spectroscopic signal of interest and a significant background level, whereas the at least second spectral components may exclusively represent the background level of the at least first spectral components.

According to a further preferred embodiment of the invention, the method further comprises the steps of at least partially transmitting at least a third spectral component of the optical signal at the first time and at least partially transmitting at least a fourth spectral component of the optical signal at the second time. Consequently, performing of the correction of the optical signal is further based on comparing the at least first, second, third and fourth transmitted and detected spectral components. In this way, the signal detected at the first time corresponds to transmission of the at least first and third spectral components of the optical signal. It is therefore a superposition of the at least first and third spectral components. Correspondingly, the second signal detected at the second time corresponds to a superposition of the at least second and fourth spectral component of the optical signal. Typically, the at least first and third spectral components refer to a spectral peak of the spectrum, whereas the at least second and fourth spectral components of the optical signal substantially refer to corresponding background level. In this way, the inventive method of correcting of the optical signal is by no means limited to the sequential acquisition of only a first and a second spectral component. Moreover, the at least first and second spectral components may constitute a plurality of different spectral bands.

In still another aspect, the invention provides a computer program product for performing a correction on an optical signal of an optical analysis system. The optical signal is spatially decomposed into its spectral components by means of a dispersive optical element. The computer program product comprises computer program means that are adapted to control spatial light manipulation means for at least partially transmitting at least a first spectral component of the optical signal at a first time and for at least partially transmitting at least a second spectral component of the optical signal at a second time. The computer program product further comprises computer program means that are adapted to process an at least first and second electrical signal for performing a correction on the optical signal. The at least first and second electrical signals are provided by a detector in response to detect the at least first and second transmitted spectral components of the optical signal.

It is further to be noted that any reference signs in the claims are not to be construed as limiting the scope of the present invention.

In the following preferred embodiments of the invention will be described in detail by making reference to the drawings in which:

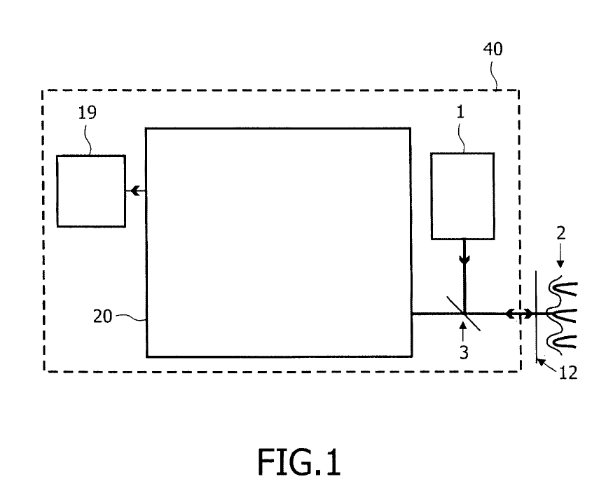

FIG. 1 is a schematic diagram of an embodiment of a blood analysis system,

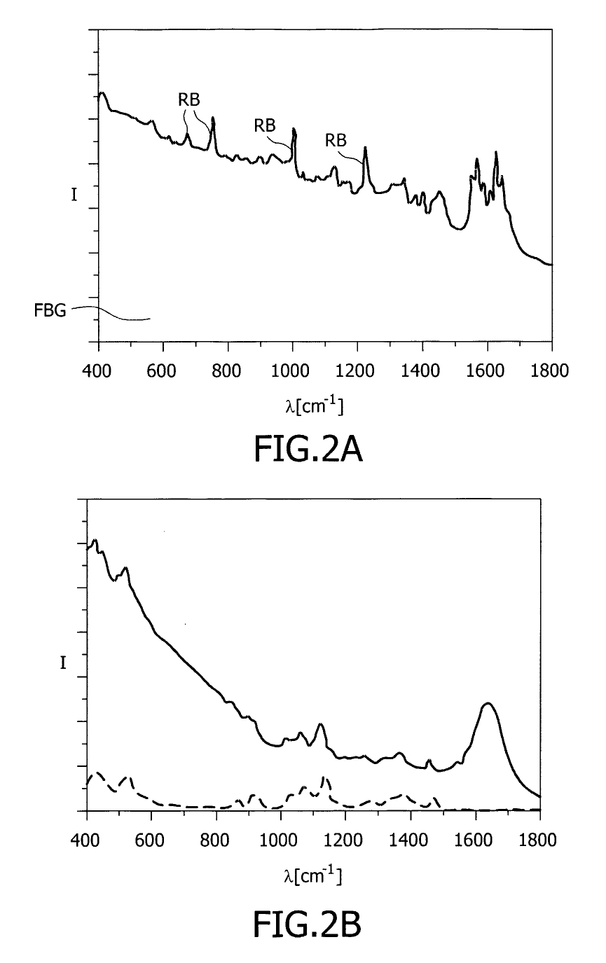

FIGS. 2A and 2B are spectra of the optical signal generated from blood in the skin and from a sample comprising one analyte in a solution,

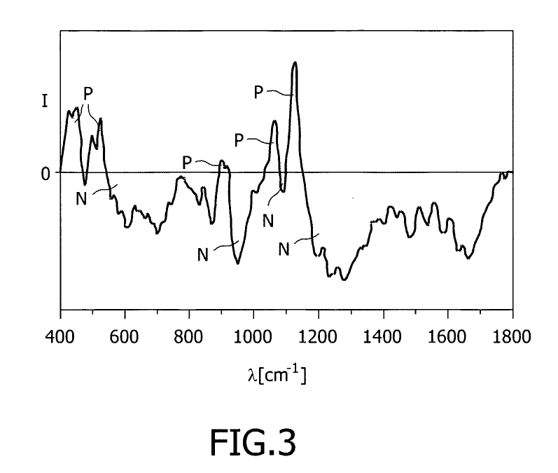

FIG. 3 is a spectral weighting function implemented in a multivariate optical element,

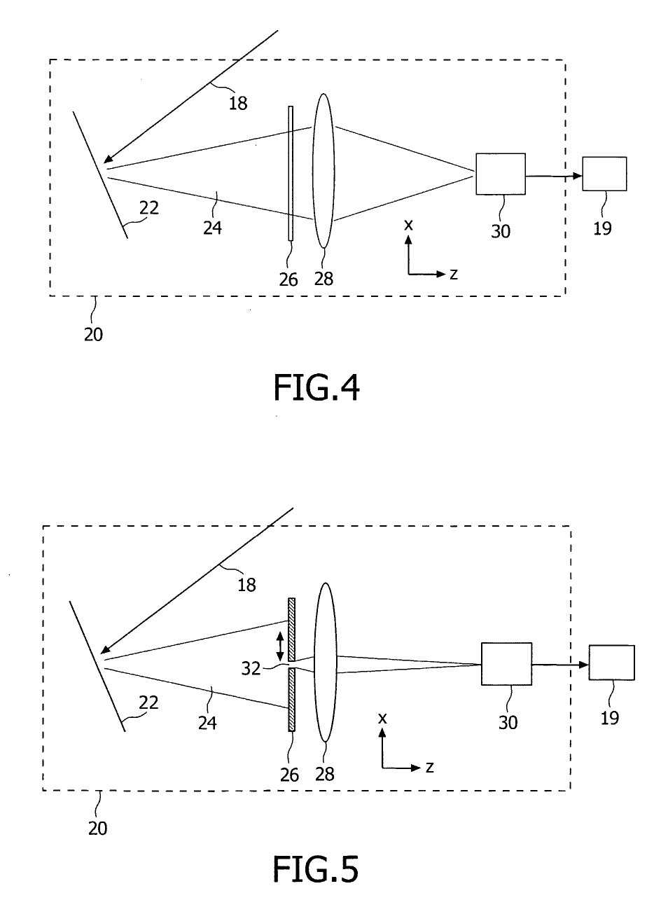

FIG. 4 shows a schematic top view illustration of the optical analysis system,

FIG. 5 schematically shows a top view illustration and a cross section of the spatial light manipulation means,

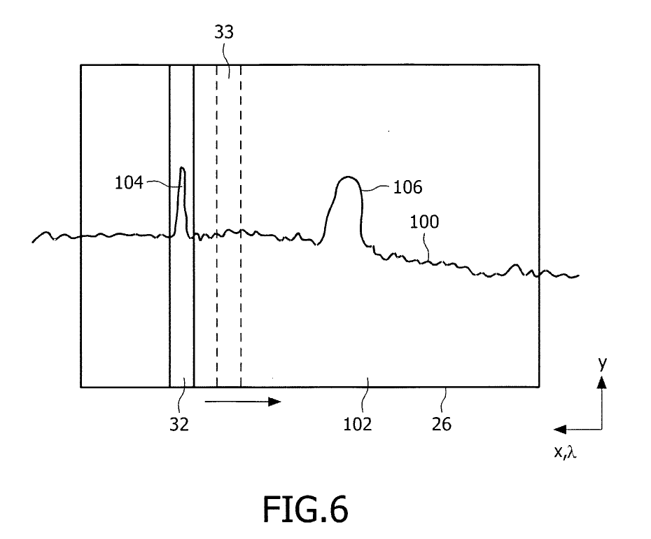

FIG. 6 shows a front view illustration of the spatial light manipulation means in combination with a spectrum,

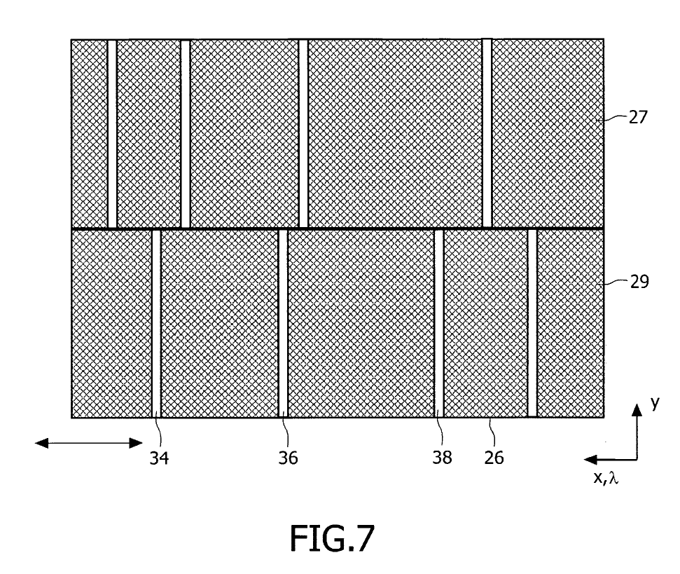

FIG. 7 shows an implementation of the spatial light manipulation means as multivariate optical element having two transmission sections.

In the embodiment shown in FIG. 1 the optical analysis system 20 for determining an amplitude of a principal component of an optical signal comprises a light source 1 for providing light for illuminating a sample 2 comprising a substance having a concentration and thereby generating the principal component. The amplitude of the principal component relates to the concentration of the substance. The light source 1 is a laser such as a gas laser, a dye laser and/or a solid state laser such as a semiconductor or diode laser.

The optical analysis system 20 is part of a blood analysis system 40. The blood analysis system further comprises a computational element 19 for determining the amplitude of the principal component, hence for determining the composition of the compound. The sample 2 comprises skin with blood vessels. The substance may be one or more of the following analytes: glucose, lactate, cholesterol, oxy-hemoglobin and/or desoxy-hemoglobin, glycohemoglobin (HbAlc), hematocrit, cholesterol (total, HDL, LDL), triglycerides, urea, albumin, creatinin, oxygenation, pH, bicarbonate and many others. The concentrations of these substances is to be determined in a non-invasive way using optical spectroscopy. To this end the light provided by the light source 1 is sent to a dichroic mirror 3 which reflects the light provided by the light source towards the blood vessels in the skin. The light may be focused on the blood vessel using an objective 12. The light may be focused in the blood vessel by using an imaging and analysis system as described in the international patent application WO 02/057759.

By interaction of the light provided by the light source 1 with the blood in the blood vessel an optical signal is generated due to Raman scattering and fluorescence. The optical signal thus generated may be collected by the objective 12 and sent to the dichroic mirror 3. The optical signal has a different wavelength than the light provided by the light source 1. The dichroic mirror is constructed such that it transmits at least a portion of the optical signal.

A spectrum 100 of the optical signal generated in this way is shown in FIG. 2A. The spectrum comprises a relatively broad fluorescence background (FBG) 102 and relatively narrow Raman bands (RB) 104, 106, 108. The x-axis of FIG. 2A denotes the wavelength shift with respect to the 785 nm of the excitation by light source 1 in wave numbers, the y-axis of FIG. 2A denotes the intensity in arbitrary units. The x-axis corresponds to zero intensity. The wavelength and the intensity of the Raman bands, i.e. the position and the height, is indicative for the type of analyte as is shown in the example of FIG. 2B for the analyte glucose which was dissolved in a concentration of 80 mMol in water. The solid line 112 of FIG. 2B shows the spectrum of both glucose and water, the dashed line 112 of FIG. 2B shows the difference between the spectrum of glucose in water and the spectrum of water without glucose. The amplitude of the spectrum with these bands is indicative for the concentration of the analyte.

Because blood comprises many compounds each having a certain spectrum which may be as complex as that of FIG. 2B, the analysis of the spectrum of the optical signal is relatively complicated. The optical signal is sent to the optical analysis system 20 according to the invention where the optical signal is analyzed by a MOE which weights the optical signal by a weighting function shown e.g. schematically in FIG. 3. The weighting function of FIG. 3 is designed for glucose in blood. It comprises a position part P and a negative part N. The positive part P and the negative part N each comprise in this example more than one spectral band.

Here and in the remainder of this application the distance between a focusing member and another optical element is defined as the distance along the optical axis between the main plane of the focusing member and the main plane of the other optical element.

A computational element 19 shown in FIG. 1 is arranged to calculate the difference between the positive and negative signal. This difference is proportional to the amplitude of the principal component of the optical signal. The amplitude of the principal component relates to the concentration of the substance, i.e. of the analyte. The relation between the amplitude and the concentration may be a linear dependence.

FIG. 4 schematically shows a top view illustration of the optical analysis system 20. The optical analysis system 20 is adapted to receive an incident optical beam 18 and to provide an electronic output to the computational element 19. The optical analysis system 20 has a grating 22 serving as a dispersive optical element, a transmission mask 26, a focusing element 28 and a detector 30. In essence, the grating 22 in combination with the transmission mask 26 serve as a multivariate optical element (MOE).

In this way dedicated spectral components of the incident optical beam 18 can be filtered and arbitrarily attenuated. By focusing the spectrally modified optical beam 18 onto the detector 30, a concentration of a particular compound of a substance can be precisely determined. The transmission pattern of the transmission mask 26 corresponds to a spectral weighting function that is specific for each compound to be analyzed by the optical analysis system 20. Typically, the detector 30 is implemented by means of a semi conductor based photodiode.

The invention effectively allows to determine the concentration of a compound without particularly performing a complete spectral analysis of the incident light beam 18. Hence, by making efficient use of the MOE, a rather expensive charge coupled device (CCD) for recording a complete spectrum 24 of the optical beam 18 can be effectively replaced by a low cost photodiode detector 30. The intensity detected by means of the detector 30 is indicative of a positive and/or negative regression function realized by the transmission mask 26. By separately detecting positive and negative parts of a spectral regression function, the concentration of a compound can be precisely determined. Therefore, the detector 30 is coupled to the computational element 19 in order to provide necessary signal processing.

Preferably, the transmission pattern of the spatial light transmission mask 26 corresponds to various spectroscopic peaks. Typically, the transmission pattern of the spatial light transmission mask 26 is realized by a plurality of slit apertures featuring a width that corresponds to the narrow spectral bands of the spectroscopic peaks, e.g. Raman bands 104, 106, 108. In a configuration where the spectroscopic peaks of the spectrum 24 exactly overlap with corresponding slit apertures of the spatial light transmission mask 26, a first signal can be detected by means of the detector 30 that comprises significant contribution from spectroscopic peaks and associate background signals.

By slightly shifting the entire spatial light transmission mask with respect to the position of the spectroscopic peaks of the spectrum 24, the spectroscopic peaks might be entirely blocked by means of the spatial light transmission mask 26 and neighboring spectral bands substantially comprising background signals are transmitted by the spatial light transmission mask 26 and detected by the detector 30. In this way two different signals are sequentially obtained allowing to extract spectroscopic information of the optical signal 18 from a superimposed first signal providing spectroscopic information as well as unavoidable broadband background.

Shifting of the entire spatial light transmission mask can in principle be performed on the basis of conventional shifting means, such like actuators based on piezo technology. Hence, a lateral displacement of the light transmission mask 26 can be electrically controlled by means of the processing means 19 that are typically implemented as a computational device, such as a personal computer. In this way, the subsequent acquisition of the at least first and second spectral components of the optical signal can be autonomously performed without manual instructions from a user.

FIG. 5 shows a top view illustration of a similar embodiment as shown in FIG. 4. Here, a cross section of the spatial light manipulation means 26 is shown. The spatial light manipulation means 26 feature an aperture 32 that effectively allows transmission of a particular spectral component of the spectrum 24. As indicated by the arrow either the entire light manipulation means 26 or the aperture 32 can be shifted in the x-direction. In this way at least first and second spectral components representing a spectroscopic peak and a background signal can be selectively transmitted and detected in a sequential way. The spatial light manipulation means 26 are by no means limited to a single aperture 32. Moreover, the mask 26 may either comprise a plurality of apertures that are fixed on the transmission mask or the transmission mask may be implemented as a reconfigurable spatial light modulator that allows to selectively provide transmission of various different spectral components.

When the aperture 32 is fixed on the mask 26, the entire mask has to be shiftable in the vertical x-direction in order to sequentially select different spectral components of the spectrum 24. Only when the light transmission mask 26 is implemented as a reconfigurable spatial light modulator, the mask 26 can be rigidly mounted with respect to e.g. the dispersive optical element. In an alternative embodiment, also the dispersive optical element 22 might be implemented as reconfigurable. For example, by modifying the orientation of the dispersive optical element 22, the spectrum 24 might be vertically shifted on the light transmission mask 26, thus effectively realizing shifting of a spectroscopic peak with respect to the aperture 32. Implementing the dispersive optical element 22 as e.g. rotatable, in principle the transmission mask 26 might also be mounted in the optical analysis system 20 in a non-moveable way.

FIG. 6 illustrates a front view of the spatial light manipulation means with a projected spectrum 100. The spectrum 100 features two spectroscopic peaks 104, 106 and a fairly uniform broad fluorescence background 102. The transmission aperture 32 is implemented as a slit aperture. As shown in FIG. 6 the horizontal width of the aperture 32 substantially matches the width of the spectral band of the spectroscopic peak 104. The spectroscopic peak 104 corresponds to the Raman band 104 as shown in FIG. 2A. When the horizontal position of the vertical slit 32 substantially matches the position of the spectroscopic peak 104, the transmitted spectral component that can be detected by the detector 30 represents a superposition of the broad fluorescence background 102 and the spectroscopic peak 104.

In order to resolve the spectroscopic contribution to the detected signal, a second signal has to be acquired that merely corresponds to the broad fluorescence background 102. This can be effectively realized by horizontally shifting the slit 32 to a position 33 as indicated by the dashed lines. In principle, the required shifting of the slit 32 can either be realized by shifting the entire mask 26 in such a way that the position of the slit 32 substantially overlaps with the indicated position 33. Alternatively, when the spatial light manipulation means 26 are implemented as a reconfigurable spatial light modulator, such as a liquid crystal light modulator, the slit 32 can be effectively moved to the position indicated by the dashed lines 33. Irrespectively of the implementation of the spatial light manipulation means 26, the second signal only represents the broad fluorescence background 102 and therefore allows to extract the spectroscopic contribution, e.g. the Raman signal of interest, of the previously acquired spectral component representing a superposition of a spectroscopic signal and a broad fluorescence background 102.

Alternatively or additionally the width of the subsequently acquired spectral bands can be arbitrarily modified. For example when the spectrum 100 features a large amount of spectral peaks, it might be advantageous to acquire a broad fluorescence spectral band on the basis of a relatively broadband selection. Therefore, the spectral width of the second transmitted spectral component may clearly deviate from the spectral width of the first acquired spectral component. In principle, by increasing the width of a slit 32, the contribution of the background signal to the acquired signal increases and the portion of the spectroscopic peaks' contribution decreases.

Also, when operating in a scanning mode, i.e. the slit 32 is horizontally moved along the entire transmission mask 26, the width of the slit 32 has a major impact on the total scanning time. The larger the slit 32 the faster a scanning can be performed. However, increasing of the slit width to an extent that is several times larger than the spectral band of a distinct spectroscopic peak does no longer allow to precisely measure the intensity of a spectral peak 104, 106. In practical implementations, it is reasonable to select a slit width that corresponds a few times the spectral bandwidth of a spectroscopic peak.

It is also reasonable to sequentially transmit and to detect adjacently located spectral bands because the broadband background might also be subject to modifications over the spectral range of the spectrum 100. Therefore, the spacing between two subsequent slit positions should be as small as possible. Otherwise the second acquired spectral component may refer to a background that strongly deviates from the background contribution of the previously acquired spectral component. Since the general structure of a spectrum 100 is principally known, selection of the at least second spectral component that is representative of broadband background can be effectively performed on the basis of the structure of the spectrum 100. For example, having knowledge that the spectrum 100 features at least two spectroscopic peaks 104, 106, it can be effectively prevented, that the second spectral component that shall represent a background signal overlaps with the peak 106.

FIG. 7 illustrates another embodiment of the transmission mask 26 featuring two transmission sections 27, 29, each of which featuring a plurality of slits 34, 36, 38. Here, each slit 34, 36, 38 corresponds to a dedicated spectral component for multivariate optical analysis. Hence, each of the slits 34, 36, 38 corresponds to the position of a spectroscopic peak in the spectrum of the incident optical signal 18. The two transmission sections 27, 29 are adapted to provide positive and negative parts of a regression function for the multivariate optical analysis, respectively. Preferably, the width of each slit 34, 36, 38 corresponds to the width of a corresponding spectroscopic peak. In a first configuration, the transmission mask 26 effectively provides transmission of those spectroscopic peaks that correspond to the horizontal position of the slits 34, 36, 38.

In this way a positive and a negative regression signal can be separately detected by means of two vertically positioned detectors. Preferably, the first detector serves to detect light being transmitted by the first transmission section 27 and the second detector is adapted to detect light being transmitted by means of the second transmission section 29. In a second configuration, the horizontal position of the slits 34, 36, 38 is slightly shifted with respect to the first configuration in order to block the corresponding spectroscopic peaks and to transmit an adjacently located spectral band being indicative of broad fluorescence background. Here, both transmission sections 27, 29 can be shifted simultaneously either by an appropriate reconfiguration of the spatial light transmission mask 26 or by shifting a fixed transmission pattern, e.g. by shifting the entire mask 26 in a horizontal direction. In this way a background signal of each positive and negative part of a regression function can be separately obtained allowing to separately correct positive and negative parts of the regression function.

The present invention therefore provides effective means for sequentially acquiring a first and a second signal that allow for background compensation of a spectroscopic signal. Preferably, the first acquired optical signal features a background and a spectroscopic contribution and the second optical signal only features a background that corresponds to the background contribution of the first signal. Selection of various spectral components of the incident optical signal 18 can be effectively performed by making use of the multivariate optical element. In particular, the sequential selection of spectroscopic and background signals can be effectively implemented into existing spectroscopic analysis systems making use of multivariate optical elements. Selection of the spectral component that is indicative of a broadband background level can be effectively realized either by reconfiguration of a spatial light transmission mask or by a slight displacement of the entire spatial light transmission mask.

List of Reference Numerals

1 light source

2 sample

3 dichroic mirror

12 objective

18 optical beam

19 computer

20 optical analysis system

22 grating

24 spectrum

26 transmission mask

27 transmission section

28 focusing element

29 transmission section

30 detector

32 slit

34 slit

36 slit

38 slit

40 blood analysis system

100 spectrum

102 broad fluorescence background

104 Raman band

106 Raman band

108 Raman band

110 combined spectrum

112 glucose spectrum

1. An optical analysis system for determining a principal component of an optical signal, the optical analysis system comprising:
a dispersive optical element for spatially separating spectral components of the optical signal in a first direction,
spatial light manipulation means for at least partially transmitting at least a first spectral component of the optical signal at a first time and for transmitting at least a second spectral component of the optical signal at a second time,
a detector for detecting the at least first and second transmitted spectral components,
processing means for performing a correction on the optical signal on the basis of the at least first and second detected spectral components.

2. An optical analysis system for determining a principal component of an optical signal, the optical analysis system comprising:
a dispersive optical element for spatially separating spectral components of the optical signal in a first direction,
a spatial light manipulator for at least partially transmitting at least a first spectral component of the optical signal at a first time and for transmitting at least a second spectral component of the optical signal at a second time,
a detector for detecting the at least first and second transmitted spectral components,
processing module for performing a correction on the optical signal on the basis of the at least first and second detected spectral components.

3. The optical analysis system according to claim 1 wherein the spatial light manipulation means is shiftable along the first direction and further comprise a fixed transmission aperture.

4. The optical analysis system according to claim 1, wherein the spatial light manipulation means comprises a reconfigurable spatial light modulator.

5. The optical analysis system according to claim 1 wherein the spatial light manipulation means further comprises an aperture having at least a first slit, the width of the at least first slit being modifiable.

6. The optical analysis system according to claim 5, wherein the spatial light manipulation means further comprises at least a second slit aperture, the at least first and second slits being simultaneously shiftable along the first direction.

7. The optical analysis system according to claim 4, the reconfigurable spatial light manipulation means forms a slit aperture moving along the first direction.

8. The optical analysis system according to claim 1 wherein the dispersive optical element and the spatial light manipulation means form a multivariate optical element, wherein the spatial light manipulation means is a liquid crystal light modulator or or a digital micro-mirror device.

9. The optical analysis system according to claim 1 wherein the system provides non-invasive analysis of blood of a person and wherein the principal component of the optical signal indicates the concentration of a substance of the blood.

10. A method of performing a correction on an optical signal of an optical analysis system, the method comprising the steps of:
spatially separating spectral components of the optical signal in a first direction by means of a dispersive optical element,
at least partially transmitting at least a first spectral component of the optical signal at a first time and at least partially transmitting at least a second spectral component of the optical signal at a second time, partially transmitting of first and second spectral components being provided by a spatial light manipulator,
detecting the at least first and second transmitted spectral components by means of a detector,
processing of the at least first and second detected spectral components for performing the correction on the optical signal.

11. The method according to claim 10, further comprising the steps of:
at least partially transmitting at least a third spectral component of the optical signal at the first time,
at least partially transmitting at least a fourth spectral component of the optical signal at the second time,
performing of the correction of the optical signal being further based on comparing the at least first, second, third and fourth transmitted spectral components.

12. A computer program product for performing a correction on an optical signal of an optical analysis system, the optical signal being spatially decomposed into its spectral components by means of a dispersive optical elemental, the computer program product comprising computer program means being adapted to:
control a spatial light manipulator for at least partially transmitting at least a first spectral component of the optical signal at a first time and for at least partially transmitting at least a second spectral component of the optical signal at a second time,
process an at least first and second electrical signal for performing a correction on the optical signal, the at least first and second electrical signals being provided by a detector in response to detect the at least first and second transmitted spectral components of the optical signal.

13. The optical analysis system according to claim 2, wherein the spatial light manipulator is shiftable along the first direction and further comprise a fixed transmission aperture.

14. The optical analysis system according to claim 1, wherein the spatial light manipulator comprises a reconfigurable spatial light modulator.

15. The optical analysis system according to claim 2, wherein the spatial light manipulator further comprises an aperture having at least a first slit, the width of the at least first slit being modifiable.

16. The optical analysis system according to claim 15, wherein the spatial light manipulator further comprises at least a second slit aperture, the at least first and second slits being simultaneously shiftable along the first direction.

17. The optical analysis system according to claim 14, the reconfigurable spatial light modulator being forms a slit aperture moving along the first direction.

18. The optical analysis system according to claim 2, wherein the dispersive optical element and the spatial light manipulator form a multivariate optical element, wherein the spatial light manipulator is a liquid crystal light modulator or a digital micro-mirror device.

19. The optical analysis system according to claim 2, wherein the system provides non-invasive analysis of blood of a person and wherein the principal component of the optical signal indicates the concentration of a substance of the blood.

20. The method of claim 10 further comprising shifting a component that determines the first spectral component of the optical signal transmitted to a location to position that allows the second spectral component of optical signal to be transmitted.
